(19) United States
(12) Patent Application Publication
(10) Pub. No.: US 2009/0130211 A1
(43) Pub. Date: May 21, 2009

(54) Gelled colloidal emulsion for appetite suppression

(76) Inventor: Aly Gamay, McLean, VA (US)

Correspondence Address: ALY GAMAY, 1102 MILL RIDGE, MCLEAN, VA, 22102, US

(21) Appl. No.: 11/984,341

(22) Filed: Nov. 16, 2007

(51) Int. Cl. A61K 36/82; A61K 9/00; A61P 43/00

(52) U.S. Cl. 424/484; 424/729

(57) ABSTRACT

Disclosed is a composition and method for producing individual dosages of chewable snack of gelled-colloidal-emulsion containing a large dose of hydrophilic and lipophilic neutraceutical and pharmaceutical preparations and in particular to the administration of edible composition having appetite reduction benefits with desirable eating characteristics for mammals.

### FIELD OF INVENTION

The present invention relates to composition and method for producing individual dosages of chewable snack of gelled-colloidal-emulsion containing large doses of hydrophilic and lipophilic neutraceutical and pharmaceutical preparations and in particular to the administration of edible composition having appetite reduction benefits with desirable eating characteristics for mammals.

### BACKGROUND

Over the past several decades the world's population, in general, has been becoming overweight. In many instances, excessive weight gain increases the risk of diabetes, hypertension, cardiac diseases or kidney disease among other diseases or conditions related to obesity. It is not just an affliction of the elderly, in fact, many recent studies show that increasing numbers of juveniles and teenagers are developing weight related afflictions.

Weight gain is a complex problem. The health conscious public is concerned about excessive weight gain, but does not generally want to maintain strict diets to lose or maintain a healthy weight.

Furthermore, weight gain is a recognizable problem in pet. The pet owning public is concerned about excessive weight of their animals, but does not generally know how to maintain a healthy weight of their dogs or cats without starving the animals.

There are many treatments and diet programs available to help promote a healthy lifestyle and weight loss. Most diet programs consist of reducing caloric intake, reducing the amount of carbohydrates consumed, and appropriate exercise. Surgical treatments are available and include stomach stapling, gastric by-pass and liposuction.

There is a need in the art for a nutritional supplement that counteracts one or more of the afore-mentioned weight related afflictions.

With the percentage of the population that is overweight, there has been a growing market for diet products. One of these types of products is appetite suppressants. A large number of these exist. Some are stimulants and others make the user nauseous. Rarely are they fully satisfactory for the typical user.

Oral dosage forms remain a significant problem for a significant segment of the population. Many individuals are unable or unwilling to swallow a solid dosage form. This problem occurs primarily in children and the elderly; however, problems with swallowing are not limited to those segments of the population. Certain conditions or disease states manifest themselves by swallowing difficulties. Otherwise healthy individuals can also exhibit problems with swallowing. Such swallowing difficulties irrespective of their cause can severely compromise patient compliance.

The neutraceutical industry has long-recognized the need for a form of oral administration, which avoids the swallowing difficulties associated with a traditional tablet. Syrups, elixirs, microcapsules containing slurries, chewable tablets and other novel tablet or capsule dosage forms have been developed, nevertheless, each has its' own disadvantages. The disadvantages include a costly process for preparation, the limitation of delivering only a small amount of active ingredients and/or more costly packaging materials.

Current offering of oral nutritional supplements in the market place for the treatment of various nutritional needs include: dry pills or capsules (requires long time for dissolving in the stomach, has questionable absorption rate, may require several or large pills) dry powders (require addition to large amount of fluids making them inconvenient to carry or consume, taste is questionable), elixirs and syrups (bulky, hard to carry, unpleasant taste has been a deterrent to broad acceptance by consumers), chewable tablets and other chews (taste is questionable, smaller dosage than needed may be delivered).

Chewable systems have been developed to deliver vitamins and other nutrients; however, they are based on low water content and low water activity for preservations. Such formulations are too firm to chew, excessively sticky to the teeth and require long time of chewing. Edible nutritional supplements that are chewable have to posses acceptable taste otherwise consumption and compliance by consumers will be affected.

An ideal weight control product will have to deliver sufficient active ingredients to suppress the appetite as well as provide a convenient and desirable way in utilization. For instance, pine nut oil is popular ingredient for weight loss; however, approximately 6000 mg have to be consumed per day. Such a high dosage represents a challenge to consumers who will have to swallow numerous large size soft gels. A novel delivery system is needed to deliver elevated concentrations of active ingredients in an easy to swallow fashion. Furthermore, soft chewable preparations, with acceptable taste and flavor, are needed to offer convenience and improve compliance of utilization.

Accordingly, there is a need for an improved nutritional supplement that can promote a healthy lifestyle and avoid drawbacks of prior art.

### SUMMARY

The present invention relates to composition and method for producing individual dosages of chewable snack of gelled emulsion containing a large dose of hydrophilic and lipophilic neutraceutical and pharmaceutical preparations and in particular to the administration of edible composition having appetite reduction benefits with desirable eating characteristics for mammals.

The method of the present invention includes the making of a composition for dispensing high dose of water soluble and fat soluble neutraceutical preparations as an emulsion in a convenient manner that does not require consumption of liquids for ingestion. The method includes providing hydrophilic and lipophilic phases of neutraceutical preparations in a delivery system that contains good tasting soft emulsion.

The method of the present invention includes methods for making a composition for dispensing high dose of hunger reducing ingredients with different physical properties in an edible format that is portable and consumable at any place.

The method of the present invention also includes methods for making a composition for dispensing high dose of hunger reducing ingredients of different mode of actions to maximize the suppression of appetite for longer duration.

A further embodiment includes a kit comprising a pre-formed package. The kit includes a gelling agent, lipid soluble additive and water soluble additive selected from the group consisting of neutraceutical compounds and mixtures thereof. The kit additionally includes the pre-formed package, which is hermetically sealed. Products are protected in hermetically sealed single dose eliminating contamination and further packaging and providing a soft texture chew.

A further embodiment includes dispensing compositions into hermetically sealed unit dose that offers portability, rigidity, formability and protection against moisture and gas extending the shelf life of unit dose even after opening the main package.

It is an object of this invention to provide a gelled-colloidal-emulsion containing large dose of hydrophilic phase and lipophilic phase of neutraceutical preparations and in particular to the administration of edible composition having appetite reduction benefits with desirable eating characteristics for mammals.

Another object of this invention can be to provide a pre-measured dosage of gelled-emulsion of appetite suppressants that is portable and has acceptable organoleptic properties to encourage compliance and provide mechanical chewing action and eating satisfying experience.

It is another object of this invention to provide a delivery system that encompasses pharmaceutical and neutraceutical preparations that does not require water to be prepared or ingested and to preserve the functionality and efficacy of compounds.

It can also be an object of the present invention, with respect to one or more of the aforementioned products/compositions, to provide a method of preparation to deliver functional food and snacks that contains large quantities of hunger reducing ingredients and vitamins.

An additional object of this invention is to provide a usable dosage of appetite suppressants without the need to consume numerous pills, soft gels or capsules.

A further object of the current invention is to provide a large dosage of appetite suppressants that is ready for instant absorption in the body and impact hunger reduction and less food consumption.

Other objects, features and advantages of the present invention will be apparent from these summary and description of preferred embodiments, and will be readily apparent to those skilled in the art having knowledge of gelled products/compositions and their methods of preparation. Such objects, features, benefits and advantages will be apparent from the above as taken in conjunction with the accompanying examples, tables, data and all reasonable inferences to be drawn there from.

### DETAILED DESCRIPTION

The present compositions include soft chews of gelled-structure preparations that contain elevated levels of fat-soluble appetite suppressants and water-soluble appetite suppressants in an edible format that does not required water consumption for ingestion.

The present method includes delivery vehicle for various water loving and lipid loving neutraceutical agents, pharmaceutical and neutraceutical preparations by the formation of compositions for treating humans and animals. The method includes heating a lipid-liking hunger reducing agent and liquid-liking hunger reducing agent, amount of water (about 15-35%) and thickening agents to about 140-190 F., and dispensing into suitable packaging materials to be cooled and allowed to solidify to produce a firm consistency.

The composition may be a gelled food with various characteristics suitable for ingestion by toddlers, young children without fear of suffocation or elderly patient with difficulties in swallowing other pharmaceutical preparations.

The components may be formulated for administration into one composition containing all the components. Alternatively, the components may be formulated into more than one composition, each of which contains one or more components. In addition, each component may constitute a separate composition.

The components may be mixed in any order to prepare the composition. The composition may comprise the same components that were added to the mixture, or any components that result from an interaction between two or more of the components after mixing.

All conditions char d to have a need for ingestible, soft and chewable delivery method for weight loss applications are able to benefit from the composition and delivery method of the invention. The composition and delivery method are effective for all mammals, including farm animals, laboratory animals, pet animals, and humans. The terms pharmaceuticals refer but not limited to pharmaceutical compounds, hormones and drugs. The terms nutraceuticals, refer to nutritional additives and nutrients including but not limited vitamins, fibers, amino acids, amino peptides, proteins, sports nutritional compounds, minerals, elements, fat-soluble oils and compounds and similar for treating conditions previously mentioned.

The composition provides satisfaction resulting from the experience of eating food in terms of mechanical action of chewing, swallowing and ingesting. That experience promotes feeling of having food consumption and satisfies hunger even though the amount of food consumed might be small in size.

The components of the composition are in a form that is systemically ingestible in an animal or human. The components employed in the method may be of various forms, consistencies or physical statuses. The nutraceuticals and pharmaceutical compounds that could be utilized in the method may be pre-hydrated, pre-solubilized, pre-coated, pre-encapsulated, microencapsulated, micronized, particulated, micro-particulated or prepared as timed-release components either individually or in various combinations.

Lipophilicity, fat-liking, refers to the ability of a chemical compound to dissolve in fats, oils, lipids, and non-polar solvents. Thus lipophilic substances tend to dissolve in other lipophilic substances. Lipophilicity, hydrophobicity and non-polarity (the latter as used to describe intermolecular interactions and not the separation of charge in dipoles) all essentially describe the same molecular attribute; the terms are often used interchangeably.

Hydrophile, refers to a physical property of a molecule that can transiently bond with water through hydrogen bonds. A hydrophilic portion of a molecule is one that is typically charge-polarized and capable of hydrogen bonding, enabling it to dissolve more readily in water than in oil or other hydrophobic solvents. Hydrophilic and hydrophobic molecules are also known as polar molecules and non-polar molecules respectively

Surfactants are compounds that are amphipathic, having a hydrophilic, water interactive ‘end’, referred to as their ‘head group’, and a lipophilic ‘end’, usually a long chain hydrocarbon fragment, referred to as their ‘tail’. They congregate at low energy surfaces, including the air-water interface (lowering surface tension) and the surfaces of the water-immiscible droplets found in emulsions (lowering interfacial tension). At these surfaces they naturally orient themselves with their head groups in water and their tails either sticking up and largely out of water (as at the air-water interface) or dissolved in the water-immiscible phase that the water is in contact with (e.g. as the emulsified oil droplet). In both these configurations the head groups strongly interact with water while the tails avoid all contact with water. Surfactant molecules also aggregate in water as micelles with their head groups sticking out and their tails bunched together. micelles draw oily substances into their hydrophobic cores, explaining the basic action of soaps and detergents used for personal cleanliness and for laundering clothes.

An emulsion is a mixture of two immiscible (unblendable) substances. One substance (the dispersed phase) is dispersed in the other (the continuous phase). Examples of emulsions include butter and mayonnaise. In butter, a continuous liquid phase surrounds droplets of water (water-in-oil emulsion). Emulsification is the process by which emulsions are prepared.

Emulsions tend to have a cloudy appearance, because the many phase interfaces (the boundary between the phases is called the interface). Emulsions are unstable and thus do not form spontaneously. Energy input through shaking, stirring, homogenizers, or spray processes are needed to form an emulsion. Over time, emulsions tend to revert to the stable state of oil separated from water. Surface active substances surfactants can increase the kinetic stability of emulsions. There are three types of emulsion instability: flocculation, where the particles form clumps; creaming, where the particles concentrate towards the surface (or bottom, depending on the relative density of the two phases) of the mixture while staying separated and breaking and coalescence where the particles coalesce and form a layer of liquid.

Emulsions are part of a more general class of two-phase systems of matter called colloids. Although the terms colloid and emulsion are sometimes used interchangeably, emulsion tends to imply that both the dispersed and the continuous phase are liquid.

A Colloid or colloidal dispersion is a type of homogenous mixture. A colloid consists of two separate phases: a dispersed phase and a continuous phase. In a colloid, the dispersed phase is made of tiny particles or droplets that are distributed evenly throughout the continuous phase. The size of the dispersed-phase particles are between 1 nm and 100 nm in at least one dimension. Homogeneous mixtures with a dispersed phase in this size range may be called colloidal aerosols, colloidal emulsions, colloidal foams, colloidal dispersions, or hydrosols. The dispersed-phase particles or droplets are largely affected by the surface chemistry present in the colloid

Because the size of the dispersed phase may be hard to measure, and because colloids look like solutions, colloids are sometimes characterized by their properties. For example, if a colloid has a solid phase dispersed in a liquid, the solid particles will not pass through a membrane, whereas the dissolved ions or molecules of a solution will pass through a membrane. In other words, dissolved components will diffuse through a membrane through which dispersed colloidal particles will not.

A gel is a colloidal system in which a porous network of interconnected nanoparticles spans the volume of a liquid medium. In general, gels are apparently solid, jelly-like materials. Both by weight and volume, gels are mostly liquid in composition and thus exhibit densities similar to liquids, however have the structural coherence of a solid. An example of a common gel is edible gelatin.

In the present invention, a gelled-colloidal-emulsion is formed to include all lipid phases and water phases in a gel like format. The lipophilic and lipophobic actives are entrapped in set and firm matrix that delivers various attributes and avoid emulsion break down.

The emulsion matrix essentially entraps various pharmaceutical and neutraceutical preparations making them available for fast disintegration in the stomach and enhanced absorption in the small intestine. The emulsion-like structure maintains the present nutraceuticals compositions ready for ingestion without the need for fluids to aid in swallowing.

The thickening and gelling agents produce gelled emulsion structure that allows for incorporation of flavors and colors. Furthermore, the gelled emulsion structure enhances the packaging and portability of finished products.

The gelling agents stabilize the emulsion without the need for surfactants or emulsifiers, even though they may be utilized in the current invention. Once the gel is cooled down to room temperature, the emulsion is stable and keeps both phases of hydrophobic and hydrophilic materials essentially blended and homogeneous.

The soft texture of the gelled emulsion structure allows for ease of consumption such that it can be a chewable snack. Thickening and gelling agents produce a viscosity greater than 1000,000,000,000 Pascal-second in the finished gelled snack. The soft snack has a set consistency that differentiates it from emulsions of runny consistency that are spoonable or drinkable in nature. The set texture allows for ease of use without spilling, dripping or leaving a trace on the hands of end users. The firm texture of the present invention allows for better handling and delivering the required concentrations of weight loss and appetite reducing ingredients.

The presence of other ingredients such as gelling agents, sweeteners, fillers, stabilizers allows for the ingestion of pharmaceuticals/nutraceuticals weight management compositions on an empty stomach without upsetting the stomach. Most of the current compositions available require not to be consumed without food or fluids.

### Weight Management Ingredients:

Water soluble (hydrophilic) and fat soluble (lipophilic) compounds could be combined in a single unit dose to offer unique composition suitable for treating various obesity conditions. For instance, a functional food for treating obesity may be produced by combining edible oils like pinolenic acid, pine nut oil, conjugated linolenic acid and water soluble materials such as Hoodia extract, liquid herbal extracts and vitamins.

Lipophilic Weight Management Ingredients:

Lipid materials in the present invention are essentially the fat portion of the emulsion. No additional fat or oils is needed to impact the formation of the emulsion. In a chocolate flavored gelled snack, the lipid materials serve as a flavor carrier and taste enhancer. The presence of these therapeutically and nutritional oils helps improve the taste of the snack, create a sensational mouth feel and mask off-taste often associated with nutritional ingredients to be chewed. Moreover, the fat soluble nutritional lipids help attain a soft texture that is easy to chew without sticking to teeth or requiring strong mechanical action.

The lipid portion of the colloidal emulsion is significantly the functional fat as well. The nutritional lipids and lipophilic materials are present at about 5 to 35%.

Pine nut oil promotes stimulation of a protein called cholecystokinin (CCK). This protein, produced in the small intestine and also present in the brain, is produced in the duodenum after eating and sends a “full” feeling to the brain. At the same time, CCK slows the rate of stomach emptying, further enhancing the feeling of satiety. Pinolenic acid, an active ingredient isolated from the genus Pinus, is a triple-unsaturated fatty acid which is a positional isomer of a more widely known gamma-linolenic acid (GLA) and is found exclusively in pine nut oil. Suitable pine nut oil extracts that contains Pinolenic acid are commercially available from, for example, Lipid Nutrition, Durkee Road 24708, Channahon, Ill., USA under the trademark PinnoThin. Derivatives of Pinolenic acid include esters, such as methyl and ethyl esters, mono-, di-, and triglycerides, salts of the carboxylic acid are acceptable. Esters can be prepared by transesterification of the carboxylic acid by techniques known in the art. Acceptable salts include alkali, alkaline, and ammonium salts and the like.

The pine nut oil contains increased concentrations of Pinolenic acid. Generally, two, three, four or more dosages of the pine nut oil are taken over the course of a day to provide between about 1000 milligrams and about 6000 milligrams that are consumed generally packed in soft gel capsules. It is preferred to use a glyceride material with a Pinolenic acid content of 5 to 50 wt %, preferably 10 to 35 wt. Generally, between about 250 milligrams and about 2000 milligrams of Pinolenic acid can be included in a composition of the invention.

Conjugated linoleic acid “CLA” may also be used herein, “CLA” encompasses a single isomer, a selected mixture of two or more isomers, and a non-selected mixture of isomers obtained from natural sources, as well as synthetic and semi synthetic CLA. CLA is an omega 6 oil. Suitable sources of CLA include, for example, sunflower oil, corn oil, or safflower oil. Typically, the oils provide a CLA content of between about 70 and about 90% (by weight), more particularly between about 75 and about 85%, and even more particularly, between about 78 and about 84% by weight. It is believed that CLA reduces body fat by enhancing insulin sensitivity so that fatty acids and glucose can pass through muscle cell membranes and away from fat tissue. This results in an improved muscle to fat ratio. Compelling evidence indicates that CLA can promote youthful metabolic function and reduce body fat. Generally, between about 250 milligrams and about 5000 milligrams of CLA can be included in a composition of the invention, in particular, between about 500 milligrams and about 1000 milligrams. Typically a composition is provided that includes between about 250 and about 1500 milligrams of the CLA.

The compositions of the invention may comprise one or more other fatty acids (i.e., straight chain carboxylic acids having from 12 to 24 carbon atoms). Examples of other fatty acids suitable for use in the present invention include linoleic acid, oleic acid, taxoleic, juniperonic, sciadonic, saturated fatty acids and EPA (eicosapentaenoic) and DHA (docosahexaenoic) (optionally as an enriched isomer mixture of EPA or DHA). Enrichment involves the alteration of the isomer mixture normally present (for example in a natural product), such as an alteration in the relative amounts of different geometrical isomers. In these compositions, the other fatty acid or each of the other fatty acids can independently be present as a free fatty acid or as a derivative thereof (including a mono-, di- or triglyceride and salts), or as a mixture thereof.

Fat soluble antioxidants such as Coenzyme Q10 may be incorporated in the emulsion to provide a dose ranging from 5 to 200 mg. In general, therapeutically lipophilic phase may be oil, solid fat, triglycerides or fatty acids and glycerides.

Hydrophilic Weight Management Ingredients:

The present invention is characterized by having sufficient water content to make it a soft chew that requires minimal chewing to be ingested. Other chews that exist on the market contain low level of water (to enhance shelf life) and that in turn results in very chewy and firm chews that stick to the teeth of end users and may limit the usage by consumers that have denture applications or have weak teeth. In the current invention, water is added at 10 to 50 of the composition to produce soft chewable product. Additionally, containing large amounts of water allows for the inclusion of liquid extracts of herbal preparations and delivers water soluble nutritional components in a manner ready for absorption in the body.

By way of example of water soluble nutraceutical compounds that may benefit form the present invention is L-Carnitine and other water soluble energy generating ingredients.

The nutraceutical components may contain appetite satiation ingredients, including, but not limited to, Hoodia Gordenii Cactus, Hoodia, Green Tea Leaf Extract, Garcinia cambogia, Hydroxycitrate (HCA), L-Carnitine, Gymnema Sylvestre, Tyrosine; fitness ingredients including, but not limited to, Chromium Picolinate.

The inclusion of large amounts of herbs such as Hoodia Gordenii Cactus or green tea may impart unpleasant taste to the finished gelled emulsion. Because of the hydrophilic nature of part of the current emulsion, Liquid extracts may be added. Liquid extracts of herbs and nutritional preparations may be delivered in large doses because the of the actual weight of finished product (about 10 to 30 grams). It would have been either difficult or impractical for other delivery methods such as capsules and soft gel to deliver effective dosage of active ingredients.

The Hoodia extract can be prepared from plant material such as the stems and roots of plants of the genus Hoodia or the genus Trichocaulon that grow in the arid regions of southern Africa. The plant extract is generally obtained from one of the species: Trichocaulon piliferum; Trichocaulon officinale; Hoodia currorii; Hoodia Gordenii; and Hoodia lugardii. Extracts from Hoodia or Trichocaulon provide steroidal glycosides, which appear to fool the brain into thinking the stomach is “full” and act as appetite suppressants. Hoodia Gordonii Cactus: Hoodia Gordonii Cactus contains a molecule that is estimated to be up to 100,000 times as potent as glucose in sending a signal to the brain that the body is in a state of satiety, or in common terms not hungry. Generally, between about 50 milligrams and about 4000 milligrams of Hoodia extract can be included in a composition of the invention, in particular, between about 500 milligrams and about 2000 milligrams on a weight basis.

Liquid herbal preparation allows for the elimination of actual dry herbal components that are not needed or impart undesirable taste or aroma to chewable nutraceuticals. Liquid herbal preparations are prepared by dissolving the selected herbs in water, heating to 160 F. and filtering the liquid to remove unneeded plant materials. Herbal extracts and of various concentrations may be employed as such or after dissolving in water and or glycerin and filtering.

Optional Nutraceuticals

The present composition and method can employ numerous types of vitamins, probiotics, enzymes, hormones, nutritional supplements synthetic compounds or other nutritional compounds and mixtures thereof in various forms and shapes.

Examples of nutraceuticals that may be employed in this method may include but not limited to: vitamins (I.E. A, B, C, D, E, K) minerals (i.e. iron, calcium, copper, zinc, chromium, potassium, phosphorus, magnesium), soluble and non soluble fiber (i.e. pectin, oat bran, Psyllium, cellulose), probiotics (i.e. Acidophilus, Bifidobacterium), enzymes (i.e. proteinase, lipase), thermogenic compounds, energy compounds, sports nutrients and other sports and anabolic compounds, nutritional material (i.e. amino acids, L-glutamine, taurine, whey proteins, animal and plant proteins, peptides), fatty acids (and derivative off) and herbal preparations (i.e. Ginseng, Echinacea, Goldenseal). The components employed in the method may be of various forms, consistencies or physical statuses. The nutraceutical compounds that could be utilized in the method may be pre-hydrated, pre-solubilized, pre-coated, pre-encapsulated, microencapsulated, micronized, particulated, and micro-particulated or prepared as timed-release components either individually or in various combinations.

### Gelling Agents

The gelling agent allows setting, firm or gelling the colloidal emulsion of various lipophobic and lipophilic portions. The resultant gelled structure allows for handling and consuming the current invention as a snack or soft chewable snack. The gelled consistency allows for strengthening the emulsion matrix and prevents the collapse of the emulsion. The gelled matrix is cohesive at room temperature, and then melts at temperature about or above 90 F., then reforms to original shape when the temperate is dropped. The gelling agent a help maintain the emulsion colloid intact without leaking out oil or fatty materials. The strength of the gelled structure may vary based on the desired final chew required but in general it has softer consistency than other low moisture chews available in the marketplace.

The present gelling agents may be selected from a member from the group consisting of collagen (gelatin), gellan gum, carbohydrate gel forming polymers (such as pectin), agar, carrageenan and alginates. Other thickening agents may include but not be limited to guar, Xanthan, Caribbean gums, Carboxymethyl cellulose and gel forming starches and carbohydrates. Gelling agent that forms an irreversible gel may also be utilized. An irreversible gel is a gel that will set quickly, but will also tend to degrade in texture and strength under conditions of increased shear and temperature.

The particular gelling agent(s) usage level depends upon a variety of factors such as the desired textural properties in the finished product, total solids level and type, and strength of the gelling agents. Generally, however, good results are obtained when the total gelling agent is present at levels ranging from about 2% to 10%.

An example gelling agent includes gelatin. Gelatin is derived from denatured collagen. Gelatin is obtained by the controlled hydrolysis from fibrous insoluble collagen. The pre-hydrated gelatin is suitable for immediate ingestion and digestion, and absorbable in the digestive tract. Gelatin offer specific advantage in terms of fast degradation in the digestive tract by the influence of digestive proteases at the body temperature.

### Optional Ingredients

Optionally, the present gel matrix products can include effective amounts of flavor(s). If present, such flavors can comprise effective amounts of flavors to provide desired flavor levels. Generally, flavors present at from about 0.01% to about 10% of the finished products are contemplated. Suitable non-nutritive sweeteners may also be used for sugar-free fictional foods. Example of non-nutritive sweeteners includes Sucralose, Aspartame, Saccharin and other high potency sweeteners. Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Example nutritive carbohydrate sweetening agents include those selected from the group consisting of sucrose, glucose, fructose, and corn syrup solids. Suitable materials for use in the current invention are those liquids and fluids with minimal amount of water (about 30% or less) and remain flowable at around 80 to 110 F. Example of such material is humectants such as glycerin (about less than 1% water, liquid at room temperature, viscous, stable, hygroscopic, clear, odorless, noncorrosive, and sweet tasting), and polylos (also referred to as sugar alcohols, part of polyols' chemical structure resembles sugar and part is similar to alcohols, the terms polyhydric alcohols and polyalcohols may also be used). Polylos group includes maltitol, sorbitol, xylitol, mannitol, isomalt and hydrogenated starch hydrolysate. Other examples of suitable media are propylene glycol, lactitol monohydrate, and erythritol. Other suitable media may include syrups of sweeteners such as maltose, fructose, glucose or natural syrups such as honey, maple syrup and corn syrup.

The present compositions can optionally contain a variety of additional ingredients suitable for rendering such products more organoleptically acceptable, more nutritious and/or more storage stable. Such optional components may include colors, coloring agents, preservatives, emulsifiers, acidity and pH modifiers (acids and alkaline). Of course, mixtures of the above-noted materials are contemplated herein.

The compositions of the present invention can be used to help manage body weight, for example to help maintain body weight at a substantially constant level or to help reduce body weight. In other words, use of the compositions can assist in slimming the body, for example by helping to induce fat loss. The use of the compositions of the invention can help to reduce calorie intake. This can help maintain body weight at a substantially constant level and/or can help reduce body weight and/or help slimming. Reduction in body weight can increase energy levels.

The use of the compositions of the invention can reduce the feeling of hunger and/or increase satiety and/or reduce the desire for high calorie foods, for example by allowing less room in the stomach for high calorie foods. In particular, the use of the compositions of the invention can enhance and/or extend satiety or fullness prior to, during and/or after a meal.

The use of the compositions of the invention can reduce the feeling of hunger during dieting and therefore increase the success rate of a diet.

Any of processing vessels may be used to combine and heat-treat the jelled product ingredients. A direct steam injection coprocessor was utilized to mix and pasteurize the gelled products before the addition of pharmaceuticals/nutraceuticals. It is also suitable to use other processing and mixing vessels with various heating/cooling, homogenizing or preparation capabilities.

Any sequence of ingredients addition may be adopted before the incorporation of nutraceuticals. In one embodiment, water is added first to the processor. Next gelling agent is added with continuous agitation. Heating is commenced to about 130° F.-190° F. The temperature of the system may be reduced to about 80-115° F. before the addition of nutraceuticals in order to minimize the detrimental impact of heat on active ingredients if needed. Fat and water soluble neutraceutical preparations, flavors, sweeteners, acidity modifiers, colors or other optional ingredients are then added.

The resultant pasteurized product has a flowable consistency suitable for further filling into suitable containers. Cooling of the finished gelled product is optional.

The gelled matrix may be filled using any of the filling equipment known to those skilled in the art of packaging technology. The gelled functional product may be dispensed in trays with cavities of the desired shape and weight, or may be filled into plastic, glass, and synthetic material, paper or like containers or packages.

The gelled functional product may be additionally dispensed into hermetically sealed packages for extended shelf life. Dispensing the compositions into hermetically sealed unit dose offers portability, rigidity, and formability. It also provides protection against moisture, gas and microbiological contamination extending the shelf life of unit dose even after opening the main package.

The gelled preparations may be handled and distributed either at room temperature, refrigerated or frozen depending on the type of pharmaceuticals/nutraceuticals compounds, distribution channels and the end-user.

### EXAMPLES

This invention is further illustrated by the following examples, which are to be regarded as illustrative only, and in no way limit the scope of the invention. The following non-limiting examples and data illustrate various aspects and features relating to the method(s) and resulting products/compositions of this invention, including the surprising and unexpected modification, control and/or improvement of the water activity level through use/incorporation of the humectants of this invention.

### Example 1

Gelled nutritional supplements were produced according to the teachings of the present invention. The gelled functional snacks were formulated using various oil soluble and herbal extracts individually or in combination. All ingredients were mixed together in a laboratory processor to make 1000 grams batches. The oil soluble and herbal extracts were added directly to the processor, and then heating took place to pasteurization to about 170 to 190 F.

The products were formulated as follows:

| | % | % | % | % | % | % |
|---|---|---|---|---|---|---|
| Ingredient | A | B | C | D | E | F |
| Gelatin | 8 | 15 | 10 | 4 | | 6 |
| Pectin | 0.75 | | | 1 | 2 | 0.2 |
| Guar Gum | 0.75 | 0.5 | | 1 | 2.5 | 0.1 |
| Mono and Di-Glycerides | 0.4 | | 0.2 | 0.4 | 0.4 | 0.4 |
| Pine Nut Oil | 20 | | 10 | 5 | 20 | 20 |
| Conjugated Linoleic Acid | | 5 | 5 | 3 | 10 | 10 |
| Docosahexaenoic | | | 5 | 3 | | 1 |
| Coenzyme Q10 | | 0.1 | | 0.1 | | 0.2 |
| Hoodia Gordenii Liquid Extract | 7 | | | 3.5 | 10 | 5 |
| Green Tea Leaf Extract | | 2 | | | | 1 |
| Hydroxycitrate | | | 2 | | | 1 |
| L-Carnitine | | | | 3.5 | | 1 |
| Chromium Picolinate | | | 0.05 | | 0.1 | 0.1 |
| Flavor | 7 | 5 | 5 | 13 | 13 | 15 |
| Corn Syrup | 40 | 20 | 10 | 35 | 10 | 10 |
| Sorbic Acid | 0.05 | 0.05 | 0.05 | 0.05 | 0.05 | 0.05 |
| Sucralose | | 0.1 | 0.25 | 0.1 | 0.2 | 0.25 |
| Vitamins | 0.28 | 0.28 | 0.28 | 0.28 | 0.28 | 0.28 |
| WATER | 15.77 | 46.67 | 52.17 | 27.07 | 31.47 | 28.42 |
| Total | 100 | 100 | 100 | 100 | 100 | 100 |

Resultant functional snacks were packaged in laminated foil pouches (20 grams each), sealed and cooled at room temperature to a set consistency. The products contained on average about 15 to 47% moisture, pH ranged between 5.0 and 6.6 and water activity (Aw) of about 0.75 to 0.95. Lipid composition ranged between 5 and 32%. The organoleptic characteristics of the functional snacks were excellent and impacted various degrees of reduction of appetite when consumed by individuals. Sample E had one of the largest impact on appetite, reducing hunger up to five hours.

### Example 2

Sugar-free gelled-colloidal-emulsion containing large doses of hydrophilic and lipophilic neutraceutical preparations were produced according to the teachings of the present invention. The emulsified functional snacks were formulated using various oil soluble and herbal extracts individually or in combination. All non active ingredients were mixed together in a laboratory processor to make 1000 grams batches. The hydrophilic and hydrophobic phases were added, and then heating took place to pasteurization to about 160 to 190 F.

The products were formulated as follows:

| | % | % | % | % | % |
|---|---|---|---|---|---|
| Ingredient | G | H | I | J | K |
| Gelatin | 6 | 12 | 10 | 3 | 8 |
| Pectin | 0.5 | | 1 | | 0.3 |
| Carrageenan | 0.5 | | 0.5 | 1 | 0.3 |
| Mono and | 0.2 | 0.3 | 0.2 | | 0.4 |
| Di-Glycerides | | | | | |
| Pine Nut Oil | 20 | | 10 | | 35 |
| Conjugated Linoleic | 10 | 20 | 10 | 1 | |
| Acid | | | | | |
| Hoodia Gordenii | 10 | | 5 | 35 | 1 |
| Liquid Extract | | | | | |
| Green Tea Leaf Extract | | 3 | | | 1 |
| Flavor | 10 | 5 | 15 | 13 | 5 |
| Citric Acid | | 0.1 | | 0.5 | |
| Glycerin | 10 | | 5 | | 5 |
| Sorbitol | 10 | 20 | 10 | 35 | 10 |
| Benzoic Acid | 0.05 | 0.05 | 0.05 | 0.05 | 0.05 |
| Aspartame | 0.3 | 0.5 | 0.4 | 0.1 | 0.8 |
| WATER | 22.45 | 39.05 | 32.85 | 11.45 | 33.15 |
| Total | 100 | 100 | 100 | 100 | 100 |

About 14 grams or the resultant functional nutrients were packaged in a multiplayer polyester film, sealed and cooled at room temperature to a set consistency. The organoleptic characteristics of the functional snacks were desirable. The products contained on average about 13 to 41% moisture, pH ranged between 4.0 and 6.7 and water activity (Aw) of about 0.70 to 0.96. The organoleptic characteristics of the functional snacks were excellent. Most of the gels had a soft but cohesive texture and soft-gelled consistency that was easy to chew without sticking to teeth. Samples impacted various degrees of hunger reduction when consumed by individuals up to five hours. After consuming the gelled-emulsioned snack, and upon eating regular food, the amount of food consumed was observed by individual to be reduced. No reports of side effect of snack were mentioned. No jittery feeling or increased heart beat were mentioned by any individual.

### Example 3

Sugar-free gelled-colloidal-emulsion containing large doses of hydrophilic and lipophilic neutraceutical preparations were produced according to the teachings of the present invention elucidated in Example 2 with the exception of replacing pine nut oil with pinolenic acid. The emulsified functional snacks were formulated using various triglycerides and herbal extracts individually or in combination.

The products were formulated as follows:

| | % | % | % |
|---|---|---|---|
| Ingredient | L | M | N |
| Gelatin | 10 | 8 | 10 |
| Carrageenan | 0.5 | | 0.1 |
| Mono and Di-Glycerides | | 0.3 | 0.2 |
| Pinolenic Acid | 3 | 6 | 6 |
| Conjugated Linoleic Acid | | 5 | 10 |
| Hoodia Powder | 3 | 1 | 5 |
| Cocoa Powder | 10 | 5 | 15 |
| Color | 0.1 | | |
| Citric Acid | | 0.05 | |
| Glycerin | 15 | 20 | 10 |
| Sorbitol | 15 | 20 | 10 |
| WATER | 43.4 | 34.65 | 33.7 |
| Total | 100 | 100 | 100 |

About 5 grams or the resultant functional nutrients were packaged in a pre-formed multiplayer polyester film, hermetically sealed and cooled to room temperature to a set consistency.

### Example 4

A kit containing sugar-free semisoft emulsion containing large doses of lipophilic and lipophobic neutraceutical preparations were produced according to the teachings of the present invention. The formulations of Example 2 were followed. The lipophilic and lipophobic nutrients were added to the gelling agents, water was incorporated, and then heating took place to pasteurization about 150 to 160 F.

About 14 grams or the resultant functional nutrients were packaged in a pre-formed multiplayer polyester film, hermetically sealed and cooled to room temperature to a set consistency. When the pre-formed package was peeled open, a firm gelled chew was released in one piece without leaving residual on fingers. When chewed, it provided a pliable and easy to chew functional food.

While the principals of this invention have been described in connection with specific embodiments, it should be understood clearly that these descriptions, along with the chosen tables and data therein, are made only by way of example and are not intended to limit the scope of this invention, in any manner. Other advantages and features of this invention will become apparent from the following claims, with the scope thereof determined by the reasonable equivalents, as understood by those skilled in the art.

What is claimed is:

1. A gelled-colloidal-emulsion composition for appetite reduction of an animal or human comprising: a gelling agent; lipophilic neutraceutical phase and hydrophilic neutraceutical phase.

2. The composition of claim 1, wherein the gelling agent is selected from the group consisting of collagen, gelatin, gellan gum, carbohydrate gel forming polymers, carrageenan, alginates, guar, xanthan, carboxymethyl cellulose, starch and mixtures or derivatives thereof.

3. The composition of claim 1, wherein the lipophilic neutraceutical phase is a triglycerides selected from the group consisting of pine nut oil, pinolenic acid, conjugated linoleic acid, eicosapentaenoic, docosahexaenoic and CoQ10 or mixtures or derivatives thereof.

4. The composition of claim 1, wherein the hydrophilic neutraceutical phase is selected from the group consisting of hoodia gordenii cactus, hoodia, green tea leaf extract, garcinia cambogia, hydroxycitrate, L-carnitine, gymnema sylvestre, tyrosine, chromium piolinate or mixtures or liquid extracts or derivatives thereof.

5. The composition of claim 1, wherein the lipophilic neutraceutical phase is included at the quantity of about 5 to 35%

6. The composition of claim 1, wherein the hydrophilic neutraceutical phase is included at the quantity of about 3 to 30%

7. The composition of claim 1, wherein the composition further comprises between about 15% to 45% water.

8. The composition of claim 1, wherein the composition further includes nutritive and non-nutritive sweeteners, colors, preservatives, emulsifiers, flavors, humectants, vitamins, minerals, fiber and hormones or mixtures or derivatives thereof.

9. The composition of claim 1, wherein the composition is a soft-chewable snack of functional food.

10. A method of making a composition for treating a human or animal comprising:
Providing a gelling agent; oil soluble additive selected from the group consisting of pine nut oil, pinolenic acid, conjugated linoleic acid, eicosapentaenoic, docosahexaenoic and CoQ10 or mixtures or derivatives thereof; and water soluble additive selected from the group consisting of hoodia gordenii cactus, hoodia, green tea leaf extract, garcinia cambogia, hydroxycitrate, L-carnitine, gymnema sylvestre, tyrosine, chromium picolinate or mixtures or liquid extracts or derivatives thereof; and combining and heating the gelling agent, oil soluble additive and water soluble additive to form the composition.

11. The method of claim 10, wherein the oil soluble additive is included at the quantity of about 5 to 35%

12. The method of claim 10, wherein the water soluble additive is included at the quantity of about 5 to 30%.

13. The method of claim 10, further comprising water content of about 15 to 45%.

14. The method of claim 10, wherein the composition is a gelled-colloidal-emulsion composition for suppressing appetite of an animal or human.

15. A kit comprising a preformed package, the kit comprising: a gelling agent; lipid soluble additive selected from the group consisting of pine nut oil, pinolenic acid, conjugated linoleic acid, eicosapentaenoic, docosahexaenoic and CoQ10 or mixtures or derivatives thereof; and water soluble additive selected from the group consisting of hoodia gordenii cactus, hoodia, green tea leaf extract, garcinia cambogia, hydroxycitrate, L-carnitine, gymnema sylvestre, tyrosine, chromium picolinate or mixtures or liquid extracts or derivatives thereof.

16. The kit of claim 15, wherein the preformed package is hermetically sealed.

17. The kit of claim 15, wherein the composition is gelled-colloidal-emulsion composition for weight management of an animal or human.

18. The kit of claim 15, wherein the gelling agent; lipid soluble additive and water soluble additive form a soft-chewable snack of functional food containing large concentration of appetite suppressing components.
